(19) United States
(12) Patent Application Publication
(10) Pub. No.: US 2007/0238713 A1
(43) Pub. Date: Oct. 11, 2007

(54) METHODS FOR PREVENTION AND TREATMENT OF CONDITIONS ARISING FROM LOCAL ESTROGEN DEFICIENCY

(75) Inventors: Michael J. GAST, West Chester, PA (US); Eileen HELZNER, Villanova, PA (US)

Correspondence Address: Pepper Hamilton LLP/Wyeth, 500 Grant Street, One Mellon Bank Center, 50th Floor, Pittsburgh, PA, 15219-2502, US

(73) Assignee: WYETH, Madison, NJ (US)

(21) Appl. No.: 11/696,917

(22) Filed: Apr. 5, 2007

Related U.S. Application Data
(60) Provisional application No. 60/789,517, filed on Apr. 5, 2006.

(51) Int. Cl. A61K 31/56; A61K 31/57

(52) U.S. Cl. 514/170

(57) ABSTRACT

The present invention relates to methods for the prevention and treatment of conditions arising from local estrogen deficiency, such as dyspareunia, vulvar atrophy, vaginal atrophy, vaginal dryness, vulvar itching, vaginal itching, vulvar burning, vaginal burning, vulvar dystrophy, atrophic vaginitis or menopausal sexual dysfunction. In some embodiments, the methods include systemic, for example oral, administration of an estrogen, such as conjugated estrogens, and a progestagen, such as MPA, contemporaneously with local administration of an estrogen, for example conjugated estrogens. In some embodiments, the methods include the oral administration of conjugated estrogens and MPA, and the vulvar, vaginal, or vulvar and vaginal administration of conjugated estrogens, for example in a cream.

This application claims the benefit of priority of U.S. Provisional Application No. 60/789,517, filed Apr. 5, 2007, which is hereby incorporated by reference in its entirety.

### FIELD OF THE INVENTION

The present invention relates to methods for the prevention and treatment of conditions arising from local estrogen deficiency. In some embodiments, the methods include systemic administration of an estrogen, such as conjugated estrogens, and a progestagen, such as medroxyprogesterone acetate (MPA), contemporaneously with local administration of an estrogen. In some embodiments, the methods include the oral administration of conjugated estrogens and medroxyprogesterone acetate, and the vulvar, vaginal, or vulvar and vaginal administration of conjugated estrogens, for example in a cream.

### BACKGROUND OF THE INVENTION

Women experience a variety of symptoms during menopause, and fewer than 25% of women actually experience a symptom-free menopausal transition. Menopausal symptoms substantially affect the quality of life (QoL) of women at this time and are often accompanied by vaginal and vulvar anatomical and physiological changes, decreased vaginal lubrication, increased vasomotor activity, cognitive changes, sleep disorders, and altered psychosocial functioning.

Atrophic vaginitis in postmenopausal women occurs due to a decline in endogenous estrogen levels. Resulting symptoms include vaginal dryness, itching, irritation and dyspareunia and can affect as many as forty percent of these women. Use of vaginal estrogen preparations for the relief of these symptoms has been proven effective, but there is little information relating to systemic absorption and resulting endometrial effects. With current FDA and medical association guidelines recommending the use of the lowest effective dose of hormone therapy for symptom relief, it is important to understand the ability of low-dose vaginal preparations to provide local relief while also analyzing their safety profiles. Studies have demonstrated the effectiveness of vaginal estrogen creams on atrophic vaginitis by directly affecting the vaginal maturation index (VMI), with evidence of improvement in VMI with a dose as low as 0.3 mg conjugated estrogens. Since publication of the results from the Women's Health Initiative (WHI) were released (Rossouw J E, Anderson G L, Prentice R L, LaCroix A Z, Kooperberg C, Stefanick M L, Jackson R D, Beresford S A, Howard B V, Johnson K C, Kotchen J M, Ockene J. Risks and benefits of estrogen plus progestin in healthy postmenopausal women: Principal results from the Women's Health Initiative randomized controlled trial. JAMA 2002; 288: 321-333.), vaginal hormone products have experienced an increase in use with menopausal women still relying on them for the local treatment of vaginal symptoms associated with menopause.

A change in sexual functioning is also a frequent concern of menopausal women and their partners. This concern is often not voluntarily shared by women with their physicians, other health care providers, or even with their colleagues or spouse. A significant body of literature, however, supports the thesis that premenopausal patterns of sexual activity and self-perceived sexuality are changed in the climacteric and beyond. Menopause is a significant risk factor for sexual dysfunction, and can be associated with detrimental effects on libido, frequency of sexual activity and vaginal dyspareunia. In addition, the menopausal transition is sometimes associated with negative changes in the women's relationship with their partner, and their ability to enjoy sexual relations.

Sexual dysfunction can play an important role in a decline in menopausal QoL for some women, and healthy sexuality may play an important role in maintaining a postmenopausal woman's overall QoL. Sexual functioning involves a complex interplay of physical and emotional factors that are influenced by the physiologic and hormonal changes that occur at this time in a woman's life. Circulating estrogen levels have been shown to be important predictors of sexual function (desire, activity, feelings/experiences and problems).

The use of hormone replacement therapy for bone loss prevention in post-menopausal women is well precedented. The normal protocol calls for estrogen supplementation using such formulations containing estrone, estriol, ethynyl estradiol or conjugated estrogens isolated from natural sources (i.e. Premarin® conjugated estrogens from Wyeth). In some patients, therapy may be contraindicated due to the proliferative effects of unopposed estrogens (estrogens not given in combination with progestins) have on uterine tissue. This proliferation is associated with increased risk for endometriosis and/or endometrial cancer. The effects of unopposed estrogens on breast tissue is less clear, but is of some concern. The need for estrogens which can maintain the bone sparing effect while minimizing the proliferative effects in the uterus and breast is evident.

Hormone therapy has been shown to produce a beneficial effect on urogenital symptoms and on the various measures of urogenital health, including vaginal cytology and endometrial thickness. However, there is little information other than the studies cited above examining the role of newer, low-dose hormone therapies on dyspareunia, sexual function and other QoL-related parameters in menopause.

A need exists for effective drug treatment for post menopausal women to relive menopausal symptoms that substantially affect the quality of life (QoL) of women. The present invention is directed to these, as well as other, important ends.

### SUMMARY OF THE INVENTION

In some embodiments, the present invention provides methods for preventing or treating a condition arising from local estrogen deficiency comprising administering systemically to a patient in need thereof:

(a) an estrogen; and

(b) a progestagen;

and contemporaneously administering locally to the patient:

(c) an estrogen.

In some embodiments, the systemically administered estrogen (a), the systemically administered progestagen (b), and the locally administered estrogen (c), are each independently administered in a continuous, intermittent or interrupted dosing regime, wherein:

the daily dose of the systemically administered estrogen is equivalent to a dose of from about 0.15 mg to about 2.5 mg of conjugated estrogens;

the daily dose of the systemically administered progestagen is equivalent to a dose of from about 0.25 mg to about 10 mg of medroxyprogesterone acetate, or from about 5 mg to about 500 mg of progesterone; and the daily dose of the locally administered estrogen is equivalent to a dose of from about 0.05 mg to about 2.5 mg of conjugated estrogens.

In some further embodiments, the estrogen (a), and the progestagen (b), are administered orally.

In some embodiments, the invention provides methods for preventing or treating a condition arising from local estrogen deficiency, comprising administering to a patient in need thereof an orally administered component and a locally administered component, wherein:

the orally administered component comprises:

(i) conjugated estrogens; and

(ii) medroxyprogesterone acetate;

and the locally administered component comprises:

(iii) conjugated estrogens;

wherein the orally administered component and the locally administered component are each independently administered in a continuous, intermittent or interrupted dosing regime, wherein:

the orally administered conjugated estrogens is administered in a dose of from about 0.15 mg to about 2.5 mg;

the orally administered medroxyprogesterone acetate is administered in a dose of from about 0.25 mg to about 10 mg; and

the locally administered conjugated estrogens is administered in a dose of from about 0.05 mg to about 2.5 mg of conjugated estrogens.

In some embodiments, the systemically administered estrogen includes or consists of conjugated estrogens. In some further embodiments, the systemically administered progestagen includes or consists of a progestin. In some further embodiments, the systemically administered progestagen includes or consists of medroxyprogesterone acetate. In some further embodiments, the systemically administered progestagen includes or consists of progesterone. In some further embodiments, the locally administered estrogen includes or consists of conjugated estrogens. In some further embodiments, the systemically administered estrogen includes or consists of conjugated estrogens; the systemically administered progestagen includes or consists of medroxyprogesterone acetate; and the locally administered estrogen includes or consists of conjugated estrogens.

In some embodiments, the locally administered estrogen is administered in one or more cream, solution, slurry, suppository, pessary, or mechanical carrier. In some embodiments, the locally administered estrogen is administered in a cream.

In some embodiments, the systemically administered estrogen, and the systemically administered progestagen, are administered in a single dosage form, for example in a tablet or capsule. In some embodiments, the single dosage form includes or consists of about 0.45 mg of conjugated estrogens, and about 1.5 mg of medroxyprogesterone acetate.

In some preferred embodiments, the orally administered conjugated estrogens is administered in a dose of about 0.45 mg; and the orally administered medroxyprogesterone acetate is administered in a dose of about 1.5 mg. In some such embodiments, the locally administered conjugated estrogens is administered in a dose of about 0.3 mg; or in a dose of about 0.45 mg.

In some further preferred embodiments, the orally administered conjugated estrogens is administered in a dose of about 0.3 mg; and the orally administered medroxyprogesterone acetate is administered in a dose of about 1.5 mg. In some such embodiments, the locally administered conjugated estrogens is administered in a dose of about 0.3 mg; or in a dose of about 0.45 mg.

In some further preferred embodiments, the orally administered conjugated estrogens is administered in a dose of about 0.625 mg; and the orally administered medroxyprogesterone acetate is administered in a dose of about 2.5 mg. In some such embodiments, the locally administered conjugated estrogens is administered in a dose of about 0.3 mg; or in a dose of about 0.45 mg.

In some embodiments, the locally administered estrogen; or the locally administered progestagen; or both the locally administered estrogen and the locally administered progestagen, is applied to the vagina, or to the vulva, or to both the vagina and the vulva. In some embodiments, the locally administered estrogen is applied in the form of a cream.

In some embodiments, the dosing regime includes or consists of daily dosing of the systemically administered estrogen, and the systemically administered progestagen; and daily dosing of the locally administered estrogen.

In some embodiments, the dosing regime includes or consists of daily dosing of the systemically administered estrogen, and the systemically administered progestagen; and intermittent or interrupted dosing of the locally administered estrogen.

In some further embodiments, the dosing regime includes or consists of intermittent or interrupted dosing of the systemically administered estrogen, and the systemically administered progestagen; and daily dosing of the locally administered estrogen.

In some further embodiments, the dosing regime includes or consists of intermittent or interrupted dosing of the systemically administered estrogen, and the systemically administered progestagen; and intermittent or interrupted dosing of the locally administered estrogen.

In some embodiments, the intermittent dosing of the systemically administered estrogen; the systemically administered progestagen; and the locally administered estrogen is each performed independently on alternate days, every third day, every fourth day, every fifth day, every sixth day or weekly.

In some embodiments, the methods of the invention are used to prevent or treat a condition arising from local estrogen deficiency that is selected from dyspareunia, vulvar atrophy, vaginal atrophy, vaginal dryness, vulvar itching, vaginal itching, vulvar burning, vaginal burning, vulvar dystrophy, atrophic vaginitis, menopausal sexual dysfunction, and atrophic changes of the vagina, vulva, bladder, bowel or other pelvic organs.

### DETAILED DESCRIPTION

The present invention provides methods for preventing or treating conditions arising from local estrogen deficiency, and particularly such conditions that substantially affect the quality of life (QoL) of women. Exemplary conditions amenable to the methods of the invention include, without limitation, dyspareunia, vulvar atrophy, vaginal atrophy, vaginal dryness, vulvar itching, vaginal itching, vulvar burning, vaginal burning, vulvar dystrophy, atrophic vaginitis, menopausal sexual dysfunction, and atrophic changes of the vagina, vulva, bladder, bowel or other pelvic organs.

In some embodiments, the methods include administering systemically to a patient in need thereof:

(a) an estrogen; and

(b) a progestagen

and contemporaneously administering locally to the patient:

(c) an estrogen.

In some embodiments, the methods of the invention utilize one or more estrogens. As used herein, the term “estrogens” is intended to include modulators that are active at the estrogen receptor, including but not limited to natural and synthetic steroidal estrogens, and natural and synthetic non-steroidal estrogens. Nonlimiting examples of estrogens useful in the methods of the invention for both systemic and local administration include estrone, estriol, equilin, estradiene, equilenin, estradiols including but not limited to ethinyl estradiol, micronized estradiol and 17 β-estradiol, 17 α-dihydroequilenin, 17 β-dihydroequilenin (See U.S. Pat. No. 2,834,712), 17 α-dihydroequilin, 17 β-dihydroequilin, menstranol and conjugated estrogenic hormones, such as those in Wyeth's Premarin® (conjugated estrogens) products. Phytoestrogens, such as equol or enterolactone, can also be used in the present methods. In some embodiments, the estrogens utilized in the invention include or consist of natural (e.g., equine) and synthetic conjugated estrogenic hormones (conjugated estrogens). One preferred example of conjugated estrogens is Wyeth's Premarin® products. Esterified estrogens, such as those sold by Solvay Pharmaceuticals, Inc. under the Estratab® trade name, may also be used with the present methods. Also useful with the methods of the invention are the salts of the applicable estrogens, most preferably the sodium salts. Examples of these preferred salts are sodium estrone sulfate, sodium equilin sulfate, sodium 17 α-dihydroequilin sulfate, sodium 17 α-estradiol sulfate, sodium delta 8,9-dehydroestrone sulfate, sodium equilenin sulfate, sodium 17 β-dihydroequilin sulfate, sodium 17 α-dihydroequilenin sulfate, sodium 17 β-estradiol sulfate, sodium 17 β-dihydroequilenin sulfate, estrone 3-sodium sulfate, equilin 3-sodium sulfate, 17 α-dihydroequilin 3-sodium sulfate, 3 β-hydroxy-estra-5(10),7-dien-17-one 3-sodium sulfate, 5-α-pregnan-3-beta-20R-diol 20-sodium sulfate, 5-α-Pregnan-3β, 16-α-diol-20-one 3-sodium sulfate, delta(8,9)-dehydroestrone 3-sodium sulfate, estra-3 β, 17 α-diol 3-sodium sulfate, 3 β-hydroxy-estr-5(10)-en-17-one 3-sodium sulfate or 5 α-Pregnan-3 β, 16 α, 20R-triol 3-sodium sulfate, or estropipate. The alkali metal salts of 8,9-dehydroestrone and the alkali metal salts of 8,9-dehydroestrone sulfate ester, as described in U.S. Pat. No. 5,210,081, which is herein incorporated by reference, also may be used. Preferred salts of estrone include, but are not limited to, the sodium and piperate salts.

In some embodiments, the systemically and locally administered estrogens include combinations of one or more suitable estrogen, such as those described above, in any combination, for example and not limitation, combinations of one or more of estradiol, estrone and estriol.

“Conjugated estrogens” (CE) as used herein includes both natural and synthetic conjugated estrogens, such as the compounds described in the United States Pharmacopia (USP 23), as well as other estrogens so considered by those skilled in the art. Although CE are typically a mixture of estrogenic components, such as estrone and equilin, the estrogens of the invention can utilize such a mixture, or include only selected or individual estrogenic components. These CE may be of synthetic or natural origin. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-alpha and beta-dihydroequilin, equilenin, 17-alpha and beta-dihydroequilenin, estrone, 17-beta-estradiol, and their sodium sulfate esters.

Naturally occurring CE are usually obtained from pregnant mare urine and then are processed and may be stabilized. Examples of such processes are set forth in U.S. Pat. Nos. 2,565,115 and 2,720,483, each of which are herein incorporated by reference.

Many CE products are commercially available. Preferred among these is the naturally occurring CE product known as Premarin® (Wyeth, Madison, N.J.). Another commercially available CE product prepared from synthetic estrogens is Cenestin® (Duramed Pharmaceuticals, Inc., Cincinnati, Ohio).

In some embodiments, the methods of the invention utilize one or more progestagens, which can be progesterone, or a progestin. Progestins known as synthetic hormones that produce effects similar to progesterone, or that have a modulating effect on progesterone, on the androgen receptors, or on both progesterone and the androgen receptors. Examples of progestagens useful in accordance with the present methods for both for both systemic and local administration include but are not limited to steroidal and non-steroidal progestins, and progesterone. Specific nonlimiting examples of useful progestagens include medroxyprogesterone acetate (MPA), norethindrone acetate (NETA), norethindrone, progesterone and micronized progesterone, levonorgestrel, gestodene, desogestrel and norgestimate.

In some embodiments, the locally administered progestagens include combinations of one or more suitable progestagens, such as those described above, in any combination.

In some embodiments, preferred systemically administered estrogens include conjugated estrogens (equine or synthetic), estradiol (micronized, or 17b-estradiol), estropipate, estradiol, estrone, estriol, ethinyl estradiol, and combinations of thereof, preferably administered orally.

In some embodiments, preferred systemically administered progestagens include medroxyprogesterone acetate, norethindrone acetate (NETA), norethindrone, progesterone (micronized), norgestimate, and combinations thereof, preferably administered orally, more preferably administered contemporaneously with the systemically administered estrogen or estrogens.

In some embodiments, preferred locally administered estrogens include conjugated estrogens (equine or synthetic), estradiol (micronized, or 17b-estradiol), estropipate, estradiol, estrone, estriol, ethinyl estradiol, and combinations of thereof, preferably administered in a cream. In some more preferred embodiments, the locally administered estrogen or estrogens include conjugated estrogens (equine or synthetic), estropipate, or estradiol, preferably in a cream, such as Wyeth's PREMARIN® (conjugated estrogens) Vaginal Cream.

Some preferred combinations of local and/or systemic estrogens/progestagens include, without limitation, estradiol/Laevo-norgestrel; 17-β estradiol/laevo-norgestrel; conjugated equine estrogens/laevo-norgestrel; estradiol/dl-norgestrel; 17-β estradiol/dl-norgestrel; estradiol valerate/laevonorgestrel; estradiol valerate/dl-norgestrel; conjugated equine estrogens/dl-norgestrel; estradiol/norethindrone (norethisteron); 17-β estradiol/norethindrone (norethisterone); estradiol valerate/norethindrone (norethisterone); conjugated equine estrogens/norethindrone (norethisterone); estradiol/norethindrone (norethisterone) acetate; 17-β estradiol/norethindrone (norethisterone) acetate; estradiol valerate/norethindrone (norethisterone) acetate; conjugated equine estrogens/norethindrone (norethisterone) acetate; estradiol/medroxyprogesterone acetate; 17-β estradiol/medroxyprogesterone acetate; estradiol valerate/medroxyprogesterone acetate; and conjugated estrogens (equine or synthetic)/medroxyprogesterone acetate.

In accordance with the methods of the invention, the systemically administered estrogen, the systemically administered progestagen, and the locally administered estrogen, can each be independently administered in a continuous, intermittent or interrupted dosing regime. The duration of the regime of any or all of the foregoing systemically or locally administered components can each independently be of any length, from a single administration, to chronic therapy regimes.

In some embodiments, the dosing regime includes or consists of daily dosing of the systemically administered estrogen, and the systemically administered progestagen; and daily dosing of the locally administered estrogen.

In some embodiments, the dosing regime includes or consists of daily dosing of the systemically administered estrogen, and the systemically administered progestagen; and intermittent or interrupted dosing of the locally administered estrogen.

In some further embodiments, the dosing regime includes or consists of intermittent or interrupted dosing of the systemically administered estrogen, and the systemically administered progestagen; and daily dosing of the locally administered estrogen.

In some further embodiments, the dosing regime includes or consists of intermittent or interrupted dosing of the systemically administered estrogen, and the systemically administered progestagen; and intermittent or interrupted dosing of the locally administered estrogen.

As used herein, the term “continuous” as used in connection with a dosing regime of the invention, is intended to mean a regime in which the dose is administered at uniform intervals, up to and including daily administration.

As used herein, the term “intermittent” as used in connection with a dosing regime of the invention, is intended to mean a regime in which the dose is administered at uniform intervals less frequently than daily. Examples of intermittent dosing regimes include alternate days, every third day, every fourth day, every fifth day, every sixth day, weekly, bi-weekly, and the like.

As used herein, the term “interrupted” as used in connection with a dosing regime of the invention, is intended to mean a regime in which the dose is administered at non-sequential or non-uniform intervals. Nonlimiting examples of interrupted dosing regimes include a period of continuous administration (e.g., daily) followed by a period of discontinuous or intermittent administration, or a period of non-administration, optionally followed by an additional period of continuous or intermittent administration, or periods in which the various components of the regimen are administered alternately in either a continuous or intermittent fashion.

As indicated above, it is contemplated that the systemically administered estrogen, the systemically administered progestagen, and the locally administered estrogen, can each be independently administered in a continuous, intermittent or interrupted regime. Thus, dosage regimes such as those in Wyeth's Premphase® products (i.e., twenty-one days of conjugated estrogens and seven days of a progestin) are amenable to the present methods.

In general, regardless of the specific dosing regime, the daily (one day) dose of the systemically (for example, orally) administered estrogen is generally equivalent to a dose of from about 0.15 mg to about 2.5 mg of conjugated estrogens; the daily dose of the systemically (for example orally) administered progestagen is equivalent to a dose of from about 0.25 mg to about 10 mg of medroxyprogesterone acetate, or from about 5 mg to about 500 mg of progesterone; and the daily dose of the locally administered estrogen is equivalent to a dose of from about 0.05 mg to about 2.5 mg of conjugated estrogens. In some preferred embodiments, the daily dose of systemically administered estrogen includes or consists of about 0.45 mg of conjugated estrogens, and the daily dose of systemically administered progestagen includes or consists of about 1.5 mg of medroxyprogesterone acetate.

As used herein, a dosage of an estrogen that is “equivalent to a dose of from about 0.15 mg to about 2.5 mg of conjugated estrogens” or “equivalent to a dose of from about 0.05 mg to about 2.5 mg of conjugated estrogens” is intended to mean a dose of such estrogen that exerts an effect upon vaginal or other pelvic tissues, or other estrogen responsive tissues or organs that is comparable to a dose of the indicated amount of conjugated estrogens, as determined by standard bioassay, immunoassay, or other analytical assay technique, in vivo or in vitro activity assay, or by any measure of clinical change such as histological change in a responsive tissue, imaging of a responsive tissue (eg. bone mineral density, breast density), or change in a biologic marker of estrogen activity, etc. Nonlimiting examples of doses of estrogens equivalent to a dose of from about 0.15 mg to about 2.5 mg of conjugated estrogens are provided below; and in U.S. Pat. No. Re. 36,247, Reissued Jul. 6, 1999, the content of which is incorporated by reference in its entirety:

| Estrogen | Equivalent Dose |
|---|---|
| Conjugated Estrogens | about 0.15 mg to about 2.5 mg |
| Estradiol | about 0.25 mg to about 2 mg |
| 17-βEstradiol | about 0.25 mg to about 2 mg |
| Estradiol Valerate | about 0.25 mg to about 2 mg |
| Estrone | about 0.15 mg to about 2.5 mg |
| Estropipate | about 0.125 mg to about 2.5 mg |
| Ethinyl Estrodiol | about 0.0025 mg to about 0.020 mg |
| Mestranol | about 0.0025 mg to about 0.030 mg |
| Quinestrol | about 0.0025 mg to about 0.020 mg |

Other nonlimiting examples of doses of estrogens equivalent to a dose of from about 0.15 mg to about 2.5 mg of conjugated estrogens may be determined by reference to U.S. Pat. Re. 36,247 after adjustment for variation in lower limit for the dose of conjugated estrogens. For example, since the lower limit of the dose of conjugated estrogens in the '247 patent was 0.3 mg rather than 0.15 mg, the lower limit of each equivalent dose has been adjusted to 50% of the value. Some of the estrogens listed below are non-steroidal estrogens. While useful in this invention, it is preferable that the non-steroidal estrogens be avoided for women who have not definitely arrived at menopause or who could become pregnant.

| Estrogen | Equivalent Dose |
|---|---|
| Piperidine estrone sulphate | about 0.125 mg to about 2.5 mg |
| Estriol | about 0.025 mg to about 0.5 mg |
| Estriol succinate | about 0.025 mg to about 0.5 mg |
| Polyestriol phosphate | about 0.025 mg to about 0.5 mg |
| Silboestrol | about 0.01 mg to about 2 mg |

| Estrogen | Equivalent Dose |
|---|---|
| Stilboestrol dipropionate | about 0.01 mg to about 2 mg |
| Diethylstilboestrol | about 0.2 mg to about 2.5 mg |
| Chlorotrianiscos | about 0.5 mg to about 2.5 mg |
| Benzoestrol | about 0.25 mg to about 2.5 mg |
| Dienoestrol | about 0.1 mg to about 2.5 mg |
| Hexoestrol | about 0.1 mg to about 2.5 mg |
| Methallenostril | about 0.25 mg to about 2.5 mg |

Those of skill in the art will appreciate that doses of the foregoing estrogean that are equivalent to a dose of from about 0.05 mg to about 2.5 mg of conjugated estrogens can be ascertained by decreasing the lower limit of the stated Equivalent Doses to one third the values stated above.

As used herein, a dosage of a progestagen that is “equivalent to a dose of from about 0.25 mg to about 10 mg of medroxyprogesterone acetate” or “equivalent to a dose of from about 5 mg to about 500 mg of progesterone” is intended to mean a dose of such progestagen that exerts an effect upon vaginal or other pelvic tissues, or other progesterone or progestin responsive tissues or organs that is comparable to a dose of the indicated amount of medroxyprogesterone acetate or progesterone, as determined by standard bioassay, immunoassay, or other analytical assay technique, in vivo or in vitro activity assay, or by any measure of clinical change such as histological change in a responsive tissue, imaging of a responsive tissue (eg. endometrial thickness, breast density), or change in a biologic marker of progesterone or progestin activity, etc. Nonlimiting examples of doses of progestagens equivalent to a dose of from about 0.25 mg to about 10 mg of medroxyprogesterone acetate (or about 5 mg to about 500 mg of progesterone) are provided below.

| Progestin | Equivalent Dose |
|---|---|
| Medroxyprogesterone acetate | about 0.25 mg to about 10 mg |
| Laeve-norgestrel | about 0.006 mg to about 0.115 mg |
| dI-noregestrel | about 0.125 mg to about 0.225 mg |
| Norethindrone (norethisterone) | about 0.004 mg to about 1.5 mg |
| Norethindrone (norethisterone) | about 0.025 mg to about 1.5 mg |
| acetate | |
| Ethynodiol diacetate | about 0.025 mg to about 1.5 mg |
| Dydrogesterone | about 1.25 mg to about 45 mg |
| Norethynodrel | about 0.05 mg to about 7.5 mg |
| Allylestrenol | about 0.25 mg to about 15 mg |
| Lynoestrenol | about 0.075 mg to about 3 mg |
| Quingestanol acetate | about 0.0125 mg to about 1.5 mg |
| Medrogestone | about 0.25 mg to about 15 mg |
| Norgestrienone | about 0.005 mg to about 0.3 mg |
| Dimethisterone | about 0.125 mg to about 23 mg |
| Ethisterons | about 0.25 mg to about 38 mg |
| Cyproterone acetate | about 0.075 mg to about 15 mg |

Other nonlimiting examples of a dosage of a progestagen that is “equivalent to a dose of from about 0.25 mg to about 10 mg of medroxyprogesterone acetate” may be extrapolated from the dosages in U.S. Pat. Re. 36,247, for example, after adjustment for the variation in the lower and upper limit for the dose of medroxyprogesterone acetate (MPA).

| Progestin | Equivalent Dose |
|---|---|
| Chlormadinone acetate | about 0.025 mg to about 0.66 mg |
| Megestrol acetate | about 0.025 mg to about 6.66 mg |

Systemic administration of estrogens and progestagens in accordance with the methods of the invention can be administered by any of a variety of routes standard in the art, including for example and not limitation orally, transdermally, via injection, via an implant, intravaginally, rectally and the like. In some embodiments, systemic administration is achieved orally via ingestion of a pill, tablet, capsule or other oral dosage form. In some embodiments, the systemically administered estrogen, and the systemically administered progestagen, are administered in a single dosage form, for example in a tablet or capsule. In some preferred embodiments, the single dosage form includes or consists of about 0.45 mg of conjugated estrogens, and about 1.5 mg of medroxyprogesterone acetate.

In some embodiments, the present methods include or consist of administering orally to a patient in need thereof an estrogen as described above, and a progestagen as described above; and contemporaneously administering locally to the patient an estrogen. In some further embodiments, the systemically administered estrogen includes or consists of conjugated estrogens; the systemically administered progestagen includes or consists of medroxyprogesterone acetate; and the locally administered estrogen includes or consists of conjugated estrogens.

In some further embodiments, the invention provides methods for preventing or treating a condition arising from local estrogen deficiency, comprising administering to a patient in need thereof an orally administered component and a locally administered component, wherein:

the orally administered component includes or consists of:

(i) conjugated estrogens; and

(ii) medroxyprogesterone acetate;

and the locally administered component includes or consists of:

(iii) conjugated estrogens;

wherein the orally administered component and the locally administered component are each independently administered in a continuous, intermittent or interrupted dosing regime, wherein:

the orally administered conjugated estrogens is administered in a dose of from about 0.15 mg to about 2.5 mg, preferably about 0.45 mg of conjugated estrogens;

the orally administered medroxyprogesterone acetate is administered in a dose of from about 0.25 mg to about 10 mg, preferably about 1.5 mg of medroxyprogesterone acetate; and the locally administered conjugated estrogens is administered in a dose of from about 0.05 mg to about 2.5 mg of conjugated estrogens.

In some preferred embodiments, the orally administered conjugated estrogens is administered in a dose of about 0.45 mg; and the orally administered medroxyprogesterone acetate is administered in a dose of about 1.5 mg. In some such embodiments, the locally administered conjugated estrogens is administered in a dose of about 0.3 mg; or in a dose of about 0.45 mg.

In some further preferred embodiments, the orally administered conjugated estrogens is administered in a dose of about 0.3 mg; and the orally administered medroxyprogesterone acetate is administered in a dose of about 1.5 mg. In some such embodiments, the locally administered conjugated estrogens is administered in a dose of about 0.3 mg; or in a dose of about 0.45 mg.

In some further preferred embodiments, the orally administered conjugated estrogens is administered in a dose of about 0.625 mg; and the orally administered medroxyprogesterone acetate is administered in a dose of about 2.5 mg. In some such embodiments, the locally administered conjugated estrogens is administered in a dose of about 0.3 mg; or in a dose of about 0.45 mg.

In some embodiments, the locally administered estrogen is applied to the vagina, or to the vulva, or to both the vagina and the vulva. The locally administered estrogen can be administered in accordance with the methods of the invention by any of a variety of routes standard in the art, including for example and not limitation, one or more cream, solution, slurry, suppository, pessary, or mechanical carrier. In some embodiments, the locally administered estrogen is administered in a cream. One example of such a cream is Wyeth's PREMARIN® (conjugated estrogens) Vaginal Cream.

Further examples of suitable systemic and local dosage forms amenable to the methods of the invention can be found in, for example, Remington's Pharmaceutical Sciences, 17th ed., Mack Publishing Company, Easton, Pa., 1985, which is incorporated herein by reference in its entirety.

The materials, methods, and examples presented herein are intended to be illustrative, and are not intended to limit the scope of the invention.

### EXAMPLES

Abbreviations are used through the Examples and are defined as follows. “Adj Mean Change” is adjusted mean change. “U-Lim” is upper limit. “L-Lim” is lower limit. “σ” is standard deviation. “Std Err.” is standard error. “CE” is conjugated estrogens. “MPA” is medroxyprogesterone acetate. “HRT” is hormone replacement therapy. “HT” is hormone therapy. “MFSQ” is McCoy Female Sexuality Questionnaire”. “BISF-W” is brief index of sexual functioning—women. “Group” is treatment group. “FSH” is serum follicle-stimulating hormone. “VMI” is vaginal maturation index. “QOL” is quality-of-life. “LOCQ” is last observation carried forward.

### Example 1

### Treatment of Dyspareunia with Daily Administration of Prempro® (0.3 Mg Conjugated Estrogens/1.5 mg Medroxyprogesterone Acetate) and Premarin® Vaginal Cream

Peri- and postmenopausal women diagnosed with vaginal dryness and/or dyspareunia are administered PREMPRO® (0.3 mg conjugated estrogens/1.5 mg medroxyprogesterone acetate) once daily on a continuous basis until these or other menopausal symptoms are no longer a therapeutic consideration. During the course of treatment, the women are also administered PREMARIN® (conjugated estrogens) Vaginal Cream on a daily or intermittent basis.

### Example 2

### Treatment of Dyspareunia with Daily Administration of Prempro® (0.45 mg Conjugated Estrogens/1.5 Mg Medroxyprogesterone Acetate) and Premarin® Vaginal Cream

Peri- and postmenopausal women diagnosed with vaginal dryness and/or dyspareunia are administered PREMPRO® (0.45 mg conjugated estrogens/1.5 mg medroxyprogesterone acetate) once daily on a continuous basis until these or other menopausal symptoms are no longer a therapeutic consideration. During the course of treatment, the women are also administered PREMARIN® (conjugated estrogens) Vaginal Cream on a daily or intermittent basis.

### Example 3

### Treatment of Dyspareunia with Daily Administration of Prempro® (0.625 mg Conjugated Estrogens/2.5 Mg Medroxyprogesterone Acetate) and Premarin® Vaginal Cream

Peri- and postmenopausal women diagnosed with vaginal dryness and/or dyspareunia are administered PREMPRO® (0.625 mg conjugated estrogens/2.5 mg medroxyprogesterone acetate) once daily on a continuous basis until these or other menopausal symptoms are no longer a therapeutic consideration. During the course of treatment, the women are also administered PREMARIN® (conjugated estrogens) Vaginal Cream on a daily or intermittent basis.

### Example 4

### Treatment of Dyspareunia with Daily Administration of Premphase® (Sequential 0.625 mg Conjugated Estrogens for 21 Days/Followed by 2.5 mg Medroxyprogesterone Acetate for 7 Days) and Premarin® Vaginal Cream

Peri- and postmenopausal women diagnosed with vaginal dryness and/or dyspareunia are administered PREMPhase® (0.625 mg conjugated estrogens/2.5 mg medroxyprogesterone acetate) once daily on a continuous basis until these or other menopausal symptoms are no longer a therapeutic consideration. During the course of treatment, the women are also administered PREMARIN® (conjugated estrogens) Vaginal Cream on a daily or intermittent basis.

### Example 5

### Prospective, Double-Blind Randomized Study of the Effect of Premarin® Vaginal Cream and Low-Dose Premarin®/MPA on Dyspareunia, Atrophic Vaginitis, Sexual Function, Quality of Life and Genital Blood Flow

Description of the Study

An outpatient, prospective, double-blind, randomized, placebo-controlled multi-center study was conducted to evaluation the effect of Premarin® Vaginal Cream (0.625 mg CE/g) and Low-Dose Premarin®/MPA (0.45 mg CE/1.5 mg MPA) on dysparenunia, atrophic vaginitis, sexual function, quality of life, and genital blood flow. The study was conducted at 25 different sites. 280 subjects planned to be enrolled in the study. 480 subjects were screened. 285 subjects were randomized, 215 completed, and 70 subjects did not complete the study. A subset of 35 subjects were enrolled in a substudy at one clinical site. Nine subjects failed to complete the substudy evaluations resulting in 26 completed substudy subjects.

Subjects were generally healthy postmenopausal women 45 to 65 years of age, inclusive. The other major inclusion criteria were: a) had a sexual partner or partners; b) has vaginal intercourse at least 2 times per month; c) finding 0% to a maximum of 10% superficial cells in the vaginal maturation index (VMI); d) Intact uterus; e) Last natural (without exogenous hormone therapy) menstrual cycle completed at least 12 consecutive months before screening with serum estradiol concentration ≦50 pg/ml. Subjects were enrolled if their last natural menstrual cycle occurred >6 months but <12 months before screening provided their serum estradiol concentration ≦50 pg/ml and their FSH level is greater than the lower limit for postmenopausal women for the given laboratory; f) Endometrial double-wall thickness not to exceed 5 mm as revealed by transvaginal ultrasound of the uterus. If endometrial thickness was >5 mm, perform endometrial biopsy. If biopsy results were normal (i.e., not indicative of hyperplasia or carcinoma), the patient was enrolled; g) In the opinion of the investigator, the patient will have a high probability for compliance and completion of the study; and h) Received signed, dated, and witnessed written informed consent.

Subjects were excluded if they had a history or active presence of the following items: a) Known or suspected estrogen-dependent neoplasia; b) Endometrial hyperplasia; c) Any malignancy with the exception of a history of basal cell carcinoma of the skin; d) Thrombophlebitis, thrombosis, or thromboembolic disorders related to estrogen use; e) Cerebrovascular accident, stroke, or transient ischemic attack; f) Neuro-ocular disorders, e.g., optic neuritis, retinal thrombosis, retinal vasculitis; g) Known hypersensitivity to estrogens, progestins, or other ingredients of Premarin/MPA or Premarin Vaginal Cream; h) Myocardial infarction or ischemic heart disease; i) Chronic renal or hepatic disease; j) Gallbladder disease (subjects who have had a cholecystectomy may be enrolled); k) Use of any estrogen-containing, progestin-containing, or androgen-containing medications within 8 weeks before screening for oral or vaginal therapy or 4 weeks before screening for transdermal therapy; l) Sexual dysfunction (i.e., prior diagnosis of primary anorgasmia or prior diagnosis of sexual arousal dysfunction).

Additionally, Active presence of the following also prevented enrollment: a) Elevated sitting blood pressure (>160 mm Hg systolic or >100 mm Hg diastolic) at the screening evaluation. Subjects taking ≦2 antihypertensive medications may be enrolled; b) Fasting triglycerides >300 mg/dL (3.39 mmol/L); c) Endocrine disease except for controlled diabetes mellitus (ie, HgA_{1c}, ≦7%, or HgA_{1c}≦the upper limit defined as good diabetic control for the laboratory used) and controlled thyroid disease; d) thrombophlebitis, thrombosis, or thromboembolic disorders; e) Known or suspected pregnancy; f) Undiagnosed abnormal genital bleeding; g) Evidence of malignant or pre-malignant changes on the prestudy mammogram; h) Uro-gynecologic surgery within the 3 months prior to the screening evaluation; i) Uro-gynecologic abnormalities or disorders that may prevent accurate evaluation of the study parameters; j) Untreated vaginal infection; k) Vaginitis other than that caused by estrogen deficiency; l) Cervical cytologic smear (e.g., Papanicolaou's smear [Pap]) report of squamous intraepithelial lesion (SIL) or greater, Cervical Intra-epithelial Neoplasia (CIN) 1 or greater, or any reported dysplasia; m) Clinically significant abnormal liver function test results (i.e., >1.5 times the upper limit of normal for the laboratory used); n) Malabsorption disorders; o) Use of an intrauterine device within the 3 months before screening; p) Use of any investigational drug within the 2 months before screening; q) Known alcohol or drug abuse; and r) Excessive smoking (>15 cigarettes per day).

The subjects were assigned randomly to one of the following two blinded treatment groups (Groups A or B):

| Treatment | Treatment Regimen | |
|---|---|---|
| Group | Vaginal Cream | Tablets |
| A | 1 g Premarin Vaginal | 1 low-dose Premarin/MPA |
| | Cream (0.625 mg CE/g), | tablet (0.45 mg CE/1.5 mg |
| | intravaginally, at | MPA), orally, daily × |
| | bedtime, × first 6 weeks* | six 28-day cycles* |
| B | 1 gram placebo cream, | 1 placebo tablet, orally, |
| | intravaginally, at | daily × six 28-day |
| | bedtime, × first 6 weeks* | cycles* |

*Local and systemic therapies are concurrent during the first six weeks of treatment.

The subjects participated for approximately seven months, which included a screening examination, followed by a 4-week pre-study diary period, immediately followed by six weeks of therapy with vaginal cream and six 28-day cycles of oral therapy.

This study evaluated the efficacy of Premarin® Vaginal Cream plus low-dose Premarin®/MPA (0.45 mg CE/1.5 mg MPA) in treating urogenital atrophy and its effect on perceived sexual experience. In the substudy, the effects of treatment on genital blood flow were evaluated. The study procedures performed at each visit are shown in the Study Flow Chart. The study efficacy evaluations are listed below.

Dyspareunia and sexual function was determined by:

a. Brief Index of Sexual Functioning—Women

b. McCoy Female Sexuality Questionnaire

c. Self-report daily diary cards recording occurrence of dyspareunia and sex-related vaginal/genital symptoms

Vaginal atrophy was determined by:

a. Vaginal cytology (for vaginal maturation index).

b. Vaginal pH.

c. Global physician assessment.

d. Self-report daily diary cards to record the symptoms of atrophic vaginitis

e. For patients in the substudy, colposcopic imaging

QOL was determined by The Women's Health Questionnaire. Genital blood flow was determined in the substudy patients by Color flow Doppler.

Study Flow Chart

| | | Day 0 | 6-Week | Cycle 3 | Cycle |
|---|---|---|---|---|---|
| | Screening | Baseline | Follow-up | Follow-up | 6 Final |
| Visit | 1 | 2 | 3 | 4 | 5 |
| Week | 1-4 | 0 | 6 | 12 | 24 |
| Medical history | X | | | | |
| Complete physical examination ² | X | | | | X |
| Brief physical examination ³ | | X | X | X | |
| Mammogram ⁴ | X | | | | |
| Gynecological examination ⁵ | X | | X | X | X |
| Pap smear | X | | | | |
| Vaginal cytology smear ⁶ | X | | X | X | X |
| Vaginal pH | X | | X | X | X |
| Serum estradiol, and FSH if required ⁷ | X | | | | |
| Laboratory studies ⁸ | X | | | | X |
| Additional Laboratory Studies ⁹ | X | | | X | X |
| Color Flow Doppler ¹⁰ | | X | | X | X |
| Colposcopic Imaging ¹⁰ | | X | | X | X |
| Vaginal Pulsatility ¹⁰ | | X | | X | X |
| Vaginal ultrasound ¹¹ | X | | | | X |
| Brief Index of Sexual Functioning | | X | | X | X |
| McCoy Female Sexuality Questionnaire | | X | | X | X |
| Women's Health Questionnaire | | X | | X | X |
| Daily Diary Card Self-Reporting ¹² | X - - - X | X - - - X | | | |
| Diary cards distributed ¹³ | X | X (Cycles | X (Cycle | X(Cycles | |
| | (Pre-Study) | 1-2) | 3) | 4-6) | |
| Study medication distributed | | X (Cycles | X (Cycle | X(Cycles | |
| | | 1-2) | 3) | 4-6) | |

¹ Screening examination occurred 4 weeks immediately prior to baseline evaluation, except when a subject must have washed out from a prohibited drug (e.g., 4 weeks for transdermal HRT or 8 weeks for oral HRT) prior to baseline evaluation.
² Included description of abnormal physical findings, sitting blood pressure, height, and weight.
³ Included description of new clinically significant findings, sitting blood pressure and weight.
⁴ Mammography was performed if not performed within past 12 months (or if the report is not in subject's clinic record).
⁵ Included global physician assessment at each visit and breast examination at the screening & final visits.
⁶ Vaginal smears were obtained from the lateral wall of the upper one-third of the vagina with a wooden spatula.
⁷ FSH greater than the lower limit for postmenopausal women was required if last natural menstrual period >6 and <12 months prior to screening.
⁸ Patients must have fasted for at least 12 hours prior to having blood drawn for these tests.
⁹ For substudy only, includeds urine cytology, free & total testosterone at screening, cycles 3 & 6.
¹⁰ For patients in the substudy only.
¹¹ If endometrial double-wall thickness was >5 mm, performed endometrial biopsy. If biopsy is normal, subject enrolled.
¹² A minimum of 3 full weeks (i.e., 21 days) of diary data must be recorded on the prestudy diary card for entry into the study; 4 weeks of completed diary data was preferred.

Safety was monitored by reports of adverse events (at all visits after informed consent has been obtained), and by means of medical history, complete physical examinations (including weight and sitting blood pressure). Additionally, the following laboratory safety studies were performed at both screening and final:

a. Hematology: Hemoglobin, hematocrit, red blood cell count, white blood cell count and platelets.

b. Blood Chemistry: Glucose, urea, creatinine, aspartate aminotransferase (AST), alanine aminotransferase (ALT), alkaline phosphatase, cholesterol and triglycerides.

Blood was also be collected visit for determination of estradiol levels in all patients at the screening visit. FSH was also determined if the last natural menstrual period was >6 months and <12 months before the screening examination.

Brief physical exams (including new clinically significant findings, weight and sitting blood pressure) were performed at visits, 2, 3 and 4. Mammography (if not performed within the 12 months prior to screening or if the report is not in the subject's clinic record), cervical Pap smear (screening) and vaginal ultrasound (at screening and final visits) were also performed as outlined in the Subject Flow Chart. If a vaginal ultrasound indicated an endometrial double-wall thickness >5 mm, an endometrial biopsy was performed.

Other laboratory examinations were performed as required to ensure adherence to the Study Inclusion and Exclusion Criteria. Treatment compliance was monitored by checks of returned medication and each patient's record of medication administration on the daily diary cards.

In a previous study of transdermal estrogen, a treatment effect of approximately 3.4 standard errors of the mean was observed on the frequency of painful intercourse. In order to detect a similar difference with 90% power, approximately 105 evaluable subjects/group were required (alpha=0.05, two-sided). All patients with available data were included in the analyses of the efficacy and efficacy-related data. The changes from baseline from the primary endpoint, change in frequency of painful intercourse, as well as secondary endpoints were analyzed with an analysis covariance with a baseline score as a covariate and center and treatment groups as factors. The primary analysis was done on the last observation carried forward (LOCF) values. The least square mean changes from baseline were reported along with the corresponding 95% confidence intervals. The analysis of the covariance was used to test within and between treatment group effects. Centers with 5 or fewer enrolled patients were combined into a single pooled center. Statistical testing was done at the two-sided alpha=0.05 level. Post hoc subgroup analyses were conducted on the efficacy data using the same statistical models as the planned.

Results

Efficacy Endpoints:

McCoy Female Sexuality Questionnaire (MFSQ)

The MFSQ is a measure of female sexual interest and functioning and was administered at baseline, visit 4, and visit 5. The hormone therapy (HT) group had a statistically significant decrease compared to placebo in the frequency of pain during intercourse in the LOCF analysis of the MFSQ. At the last visit, there was a statistically significant increase in the HT group versus placebo in the level of sexual interest, frequency of orgasm, and pleasure of orgasm (Tables 1-3, respectively). There was no effect of HT use on the frequency of sexual activity, frequency of sexual thoughts, excitement/arousal during sexual activity, enjoyment of sexual activity, and insufficient vaginal lubrication compared to placebo (see Table 21 for the analysis of the covariance for the MFSQ).

TABLE 1

| | | | | | Adjusted | 95% confidence | | | Within | Between |
|---|---|---|---|---|---|---|---|---|---|---|
| | | | Baseline | | Mean | Std. | | | Group | Group |
| Cycle | Group | N | Mean | σ | Change | Err. | L-Lim | U-Lim | P-value | P-value |
| Baseline | Placebo | 135 | 3.0 | 1.6 | | | | | | |
| | Premarin | 136 | 3.3 | 1.7 | | | | | | |
| Cycle 3 | Placebo | 109 | 3.0 | 1.6 | 0.2 | 0.1 | −0.0 | 0.5 | .094 | |
| | Premarin | 103 | 3.2 | 1.7 | 0.5 | 0.1 | 0.3 | 0.8 | .000 | .084 |
| Cycle 6 | Placebo | 114 | 3.0 | 1.6 | 0.2 | 0.1 | −0.1 | 0.4 | .216 | |
| | Premarin | 107 | 3.3 | 1.7 | 0.7 | 0.1 | 0.5 | 1.0 | .000 | .001 |
| LOCF | Placebo | 117 | 3.0 | 1.6 | 0.1 | 0.1 | −0.1 | 0.4 | .244 | |
| | Premarin | 111 | 3.2 | 1.7 | 0.8 | 0.1 | 0.5 | 1.0 | .000 | .000 |

TABLE 2

| | | | | | Adjusted | | | | Within | Between |
|---|---|---|---|---|---|---|---|---|---|---|
| | | | Baseline | | Mean | Std. | 95% confidence | | Group | Group |
| Cycle | Group | N | Mean | σ | Change | Err. | L-Lim | U-Lim | P-value | P-value |
| Baseline | Placebo | 134 | 3.9 | 1.9 | | | | | | |
| | Premarin | 137 | 3.9 | 2.1 | | | | | | |
| Cycle 3 | Placebo | 107 | 4.0 | 1.9 | 0.2 | 0.2 | −0.1 | 0.5 | .221 | |
| | Premarin | 102 | 3.9 | 2.1 | 0.6 | 0.2 | 0.3 | 1.0 | .000 | .060 |
| Cycle 6 | Placebo | 113 | 3.9 | 1.9 | 0.1 | 0.2 | −0.2 | 0.4 | .577 | |
| | Premarin | 104 | 3.9 | 2.1 | 0.5 | 0.2 | 0.2 | 0.9 | .002 | .042 |
| LOCF | Placebo | 115 | 3.9 | 1.9 | 0.1 | 0.2 | −0.3 | 0.4 | .713 | |
| | Premarin | 109 | 3.9 | 2.1 | 0.5 | 0.2 | 0.2 | 0.9 | .002 | .030 |

TABLE 3

| | | | | | Adjusted | | | | Within | Between |
|---|---|---|---|---|---|---|---|---|---|---|
| | | | Baseline | | Mean | Std. | 95% confidence | | Group | Group |
| Cycle | Group | N | Mean | σ | Change | Err. | L-Lim | U-Lim | P-value | P-value |
| Baseline | Placebo | 127 | 4.0 | 2.0 | | | | | | |
| | Premarin | 124 | 4.1 | 2.3 | | | | | | |
| Cycle 3 | Placebo | 97 | 4.0 | 2.0 | 0.4 | 0.2 | −0.0 | 0.8 | .052 | |
| | Premarin | 93 | 4.0 | 2.3 | 0.5 | 0.2 | 0.1 | 0.8 | .020 | .774 |
| Cycle 6 | Placebo | 101 | 4.0 | 1.9 | 0.0 | 0.2 | −0.3 | 0.4 | .797 | |
| | Premarin | 92 | 3.9 | 2.3 | 0.8 | 0.2 | 0.4 | 1.1 | .000 | .005 |
| LOCF | Placebo | 103 | 4.1 | 1.9 | 0.0 | 0.2 | −0.3 | 0.4 | .908 | |
| | Premarin | 99 | 4.0 | 2.3 | 0.6 | 0.2 | 0.2 | 1.0 | .001 | .018 |

Daily Diary Cards

Subjects maintained diary cards, which recorded the occurrence of dyspareunia and sex related vaginal/genital symptoms. The diary cards were used for the entire study. In the LOCF analysis of diary cards, the HT group had a statistically significant improvement in average pain severity per intercourse compared to placebo (Table 4). HT subjects also had a statistically significant improvement in average vaginal dryness severity perintercourse as compared to placebo (Table 5). There was no effect of HT use on the frequency of intercourse per cycle compared to placebo.

TABLE 4

| | | | | | Adj | | | | Within | Between |
|---|---|---|---|---|---|---|---|---|---|---|
| | | | Baseline | | Mean | Std | 95% confidence | | Group | Group |
| Cycle | Group | N | Mean | σ | Change | Err. | L-Lim | U-Lim | P-value | P-value |
| Pre-study | Placebo | 136 | 0.78 | 0.93 | | | | | | |
| | Premarin | 138 | 0.78 | 0.88 | | | | | | |
| Cycle 1 | Placebo | 118 | 0.76 | 0.94 | −0.28 | 0.05 | −0.38 | −0.18 | .000 | |
| | Premarin | 122 | 0.80 | 0.88 | −0.47 | 0.05 | −0.57 | −0.37 | .000 | .004 |
| Cycle 2 | Placebo | 107 | 0.79 | 0.96 | −0.36 | 0.05 | −0.46 | −0.26 | .000 | |
| | Premarin | 115 | 0.81 | 0.89 | −0.58 | 0.05 | −0.68 | −0.48 | .000 | .001 |
| Cycle 3 | Placebo | 104 | 0.77 | 0.95 | −0.26 | 0.06 | −0.37 | −0.14 | .000 | |
| | Premarin | 106 | 0.78 | 0.86 | −0.49 | 0.06 | −0.61 | −0.37 | .000 | .004 |
| Cycle 4 | Placebo | 101 | 0.73 | 0.93 | −0.27 | 0.06 | −0.38 | −0.16 | .000 | |
| | Premarin | 94 | 0.79 | 0.87 | −0.55 | 0.06 | −0.66 | −0.43 | .000 | .000 |
| Cycle 5 | Placebo | 99 | 0.76 | 0.94 | −0.24 | 0.06 | −0.36 | −0.13 | .000 | |
| | Premarin | 91 | 0.83 | 0.86 | −0.54 | 0.06 | −0.66 | −0.42 | .000 | .000 |
| Cycle 6 | Placebo | 99 | 0.77 | 0.93 | −0.28 | 0.06 | −0.39 | −0.16 | .000 | |
| | Premarin | 86 | 0.82 | 0.84 | −0.56 | 0.06 | −0.69 | −0.44 | .000 | .000 |
| LOCF | Placebo | 121 | 0.78 | 0.96 | −0.27 | 0.06 | −0.39 | −0.16 | .000 | |
| | Premarin | 126 | 0.79 | 0.87 | −0.47 | 0.06 | −0.58 | −0.36 | .000 | .010 |

TABLE 5

| | | | | | Adjusted | | | | Within | Between |
|---|---|---|---|---|---|---|---|---|---|---|
| | | | Baseline | | Mean | Std | 95% confidence | | Group | Group |
| Cycle | Group | N | Mean | σ | Change | Err. | L-Lim | U-Lim | P-value | P-value |
| Prestudy | Placebo | 138 | 1.11 | 0.97 | | | | | | |
| | Premarin | 138 | 1.02 | 0.97 | | | | | | |
| Cycle 1 | Placebo | 121 | 1.12 | 0.97 | −0.58 | 0.06 | −0.69 | −0.47 | .000 | |
| | Premarin | 123 | 1.03 | 0.98 | −0.79 | 0.06 | −0.90 | −0.68 | .000 | .005 |
| Cycle 2 | Placebo | 109 | 1.09 | 0.96 | −0.74 | 0.05 | −0.84 | −0.64 | .000 | |
| | Premarin | 115 | 1.03 | 1.00 | −0.91 | 0.05 | −1.00 | −0.81 | .000 | .010 |
| Cycle 3 | Placebo | 105 | 1.08 | 0.96 | −0.61 | 0.06 | −0.73 | −0.49 | .000 | |
| | Premarin | 107 | 1.05 | 0.99 | −0.76 | 0.06 | −0.89 | −0.64 | .000 | .069 |
| Cycle 4 | Placebo | 102 | 1.06 | 0.96 | −0.54 | 0.06 | −0.66 | −0.42 | .000 | |
| | Premarin | 94 | 1.07 | 1.01 | −0.78 | 0.06 | −0.90 | −0.65 | .000 | .003 |
| Cycle 5 | Placebo | 101 | 1.09 | 0.96 | −0.53 | 0.07 | −0.66 | −0.40 | .000 | |
| | Premarin | 91 | 1.10 | 1.01 | −0.82 | 0.07 | −0.96 | −0.68 | .000 | .001 |
| Cycle 6 | Placebo | 100 | 1.10 | 0.94 | −0.51 | 0.07 | −0.65 | −0.38 | .000 | |
| | Premarin | 87 | 1.06 | 1.00 | −0.85 | 0.07 | −0.99 | −0.70 | .000 | .000 |
| LOCF | Placebo | 123 | 1.12 | 0.97 | −0.53 | 0.06 | −0.65 | −0.41 | .000 | |
| | Premarin | 126 | 1.02 | 0.98 | −0.81 | 0.06 | −0.93 | −0.69 | .000 | .001 |

Brief Index of Sexual Functioning—Women (BISF-W)

This 22-item questionnaire is an inventory of sexual interest, activity, satisfaction and preference and was administered at baseline, visit 4, and visit 5. For the BISF-W, in the LOCF analysis, the HT group had a significant improvement in receptivity/initiation (Table 6) and relationship satisfaction (Table 7) compared to placebo. There was no difference from placebo for the other areas of the BISF-W: thoughts/desire, arousal, frequency of sexual activity, pleasure/orgasm, and problems affecting sexual function (see Table 22 for the analysis of variance for the BISF-W).

TABLE 6

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 129 | 8.45 | 2.76 | | | | | | |
| | Premarin | 131 | 8.78 | 2.84 | | | | | | |
| Cycle 3 | Placebo | 90 | 8.34 | 2.81 | 0.15 | 0.23 | −0.30 | 0.60 | .504 | |
| | Premarin | 96 | 8.68 | 2.72 | 0.69 | 0.22 | 0.26 | 1.12 | .002 | .074 |
| Cycle 6 | Placebo | 100 | 8.58 | 2.70 | −0.29 | 0.24 | −0.77 | 0.18 | .224 | |
| | Premarin | 94 | 8.57 | 2.67 | 0.61 | 0.25 | 0.11 | 1.11 | .018 | .008 |
| LOCF | Placebo | 105 | 8.47 | 2.80 | −0.19 | 0.23 | −0.65 | 0.27 | .423 | |
| | Premarin | 103 | 8.68 | 2.69 | 0.50 | 0.24 | 0.02 | 0.97 | .039 | .033 |

TABLE 7

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 133 | 7.58 | 2.70 | | | | | | |
| | Premarin | 135 | 7.73 | 2.81 | | | | | | |
| Cycle 3 | Placebo | 103 | 7.58 | 2.71 | 0.04 | 0.24 | −0.44 | 0.52 | .869 | |
| | Premarin | 99 | 7.40 | 2.87 | 0.66 | 0.25 | 0.16 | 1.15 | .010 | .062 |
| Cycle 6 | Placebo | 111 | 7.45 | 2.75 | 0.19 | 0.21 | −0.23 | 0.61 | .379 | |
| | Premarin | 100 | 7.68 | 2.82 | 1.13 | 0.24 | 0.66 | 1.60 | .000 | .002 |
| LOCF | Placebo | 112 | 7.48 | 2.76 | 0.17 | 0.21 | −0.26 | 0.59 | .437 | |
| | Premarin | 107 | 7.59 | 2.84 | 1.18 | 0.23 | 0.74 | 1.63 | .000 | .001 |

Vaginal Cytology (for Vaginal Maturation Index)

As a marker of vaginal atrophy, vaginal cytology smears were obtained from the lateral wall of the upper one-third of the vagina at the screening visit and visits 3, 4, and 5. In the LOCF analysis, the HT group had a statistically significant increase versus placebo in the percentage of superficial and intermediate cells (Tables 10 and 8, respectively) and a statistically significant decrease in the percentage of parabasal cells (Table 9).

TABLE 8

| | | | | | Adj | | | | Within | Between |
|---|---|---|---|---|---|---|---|---|---|---|
| Actual | | | Baseline | | Mean | Std | 95% confidence | | Group | Group |
| Event | Group | N | Mean | σ | Change | Err. | L-Lim | U-Lim | P-value | P-value |
| Screen | Placebo | 140 | 63.5 | 41.2 | | | | | | |
| | Premarin | 143 | 61.1 | 40.8 | | | | | | |
| 6-WK F-U | Placebo | 120 | 64.1 | 41.2 | 7.7 | 3.7 | 0.4 | 15.0 | .038 | |
| | Premarin | 119 | 58.9 | 40.8 | 9.0 | 3.7 | 1.8 | 16.3 | .015 | .737 |
| Cycle 3 | Placebo | 114 | 64.2 | 41.3 | 5.4 | 3.6 | −1.6 | 12.5 | .131 | |
| | Premarin | 108 | 61.5 | 40.5 | 14.0 | 3.9 | 6.4 | 21.6 | .000 | .040 |
| Cycle 6 | Placebo | 119 | 64.5 | 41.1 | −2.8 | 3.9 | −10.5 | 4.9 | .472 | |
| | Premarin | 109 | 62.2 | 40.3 | 12.2 | 4.1 | 4.1 | 20.3 | .004 | .001 |
| LOCF | Placebo | 121 | 65.2 | 40.9 | −2.5 | 3.8 | −9.9 | 4.9 | .507 | |
| | Premarin | 119 | 60.5 | 40.4 | 12.7 | 3.9 | 5.1 | 20.3 | .001 | .000 |

TABLE 9

| | | | | | Adj | | | | Within | Between |
|---|---|---|---|---|---|---|---|---|---|---|
| Actual | | | Baseline | | Mean | Std | 95% confidence | | Group | Group |
| Event | Group | N | Mean | σ | Change | Err. | L-Lim | U-Lim | P-value | P-value |
| Screening | Placebo | 140 | 35.0 | 41.9 | | | | | | |
| | Premarin | 143 | 36.9 | 41.5 | | | | | | |
| 6-WK F-U | Placebo | 120 | 34.2 | 42.0 | −13.4 | 2.9 | −19.1 | −7.7 | .000 | |
| | Premarin | 119 | 38.9 | 41.6 | −35.5 | 2.9 | −41.2 | −29.8 | .000 | .000 |
| Cycle 3 | Placebo | 114 | 34.0 | 42.1 | −8.1 | 3.2 | −14.5 | −1.7 | .013 | |
| | Premarin | 108 | 36.1 | 41.2 | −30.9 | 3.5 | −37.8 | −24.1 | .000 | .000 |
| Cycle 6 | Placebo | 119 | 33.8 | 41.9 | −5.8 | 3.1 | −12.0 | 0.4 | .065 | |
| | Premarin | 109 | 35.6 | 40.9 | −30.0 | 3.3 | −36.6 | −23.4 | .000 | .000 |
| LOCF | Placebo | 121 | 33.1 | 41.6 | −5.8 | 3.1 | −11.9 | 0.2 | .060 | |
| | Premarin | 119 | 37.2 | 41.2 | −29.6 | 3.2 | −35.8 | −23.3 | .000 | .000 |

TABLE 10

| | | | | | Adj | | | | Within | Between |
|---|---|---|---|---|---|---|---|---|---|---|
| Actual | | | Baseline | | Mean | Std | 95% confidence | | Group | Group |
| Event | Group | N | Mean | σ | Change | Err. | L-Lim | U-Lim | P-value | P-value |
| Screening | Placebo | 140 | 1.5 | 6.9 | | | | | | |
| | Premarin | 143 | 2.0 | 9.7 | | | | | | |
| 6-WK F-U | Placebo | 120 | 1.7 | 7.4 | 6.3 | 2.7 | 1.0 | 11.7 | .021 | |
| | Premarin | 119 | 2.3 | 10.5 | 25.5 | 2.7 | 20.1 | 30.8 | .000 | .000 |
| Cycle 3 | Placebo | 114 | 1.8 | 7.6 | 1.8 | 2.0 | −2.2 | 5.7 | .387 | |
| | Premarin | 108 | 2.4 | 11.0 | 16.2 | 2.2 | 12.0 | 20.5 | .000 | .000 |
| Cycle 6 | Placebo | 119 | 1.7 | 7.5 | 5.9 | 1.7 | 2.5 | 9.3 | .001 | |
| | Premarin | 109 | 2.3 | 10.9 | 14.0 | 1.8 | 10.4 | 17.5 | .000 | .000 |
| LOCF | Placebo | 121 | 1.7 | 7.4 | 5.3 | 1.8 | 1.7 | 8.9 | .004 | |
| | Premarin | 119 | 2.3 | 10.5 | 13.5 | 1.9 | 9.8 | 17.1 | .000 | .000 |

Vaginal pH

Vaginal pH was collected at the screening visit and visits 3, 4, and 5. The HT group had a statistically significant decrease in vaginal pH versus placebo in the LOCF analysis (Table 11).

TABLE 11

| | | | | | Adj | | | | Within | Between |
|---|---|---|---|---|---|---|---|---|---|---|
| | | | Baseline | | Mean | Std | 95% confidence | | Group | Group |
| Period | Group | N | Mean | σ | Change | Err. | L-Lim | U-Lim | P-value | P-value |
| Screening | Placebo | 141 | 5.8 | 0.9 | | | | | | |
| | Premarin | 144 | 5.9 | 1.0 | | | | | | |
| 6-WK F-U | Placebo | 121 | 5.8 | 0.9 | −0.2 | 0.1 | −0.4 | −0.0 | .015 | |
| | Premarin | 119 | 6.0 | 1.0 | −0.7 | 0.1 | −0.9 | −0.5 | .000 | .000 |
| Cycle 3 | Placebo | 115 | 5.8 | 0.9 | −0.1 | 0.1 | −0.3 | 0.1 | .248 | |
| | Premarin | 108 | 5.9 | 1.0 | −0.6 | 0.1 | −0.8 | −0.4 | .000 | .000 |
| Cycle 6 | Placebo | 120 | 5.8 | 0.9 | −0.1 | 0.1 | −0.3 | 0.1 | .283 | |
| | Premarin | 110 | 5.9 | 1.0 | −0.6 | 0.1 | −0.8 | −0.5 | .000 | .000 |
| LOCF | Placebo | 126 | 5.8 | 0.9 | −0.1 | 0.1 | −0.3 | 0.0 | .159 | |
| | Premarin | 122 | 5.9 | 1.0 | −0.6 | 0.1 | −0.8 | −0.4 | .000 | .000 |

Global Physician Assessment

A global physician assessment (physical exam) was completed at each visit. As assessed by the examining physician, the HT group had a statistically significant improvement (in the LOCF analysis) in mucosa color and rugosity compared to placebo (Tables 12 and 13, respectively). Additionally, a larger number of women in the HT group (56.6%) versus the placebo group (42.1%) were assessed as having normal, non-friable vaginal mucosa at the final visit (Table 14).

TABLE 12

| | | | | | Adj | | | | Within | Between |
|---|---|---|---|---|---|---|---|---|---|---|
| Actual | | | Baseline | | Mean | Std | 95% confidence | | Group | Group |
| Event | Group | N | Mean | σ | Change | Err. | L-Lim | U-Lim | P-value | P-value |
| Screening | Placebo | 141 | 2.3 | 0.6 | | | | | | |
| | Premarin | 144 | 2.3 | 0.5 | | | | | | |
| 6-WK F-U | Placebo | 121 | 2.3 | 0.6 | 0.3 | 0.1 | 0.2 | 0.5 | .000 | |
| | Premarin | 120 | 2.3 | 0.5 | 0.6 | 0.1 | 0.4 | 0.7 | .000 | .001 |
| Cycle 3 | Placebo | 115 | 2.3 | 0.6 | 0.2 | 0.1 | 0.1 | 0.3 | .004 | |
| | Premarin | 109 | 2.3 | 0.5 | 0.5 | 0.1 | 0.4 | 0.7 | .000 | .000 |
| Cycle 6 | Placebo | 120 | 2.3 | 0.6 | 0.3 | 0.1 | 0.2 | 0.4 | .000 | |
| | Premarin | 110 | 2.2 | 0.5 | 0.5 | 0.1 | 0.4 | 0.7 | .000 | .001 |
| LOCF | Placebo | 126 | 2.3 | 0.6 | 0.3 | 0.1 | 0.2 | 0.4 | .000 | |
| | Premarin | 122 | 2.2 | 0.5 | 0.5 | 0.1 | 0.4 | 0.7 | .000 | .004 |

TABLE 13

| | | | | | Adj | | | | Within | Between |
|---|---|---|---|---|---|---|---|---|---|---|
| Actual | | | Baseline | | Mean | Std | 95% confidence | | Group | Group |
| Event | Group | N | Mean | σ | Change | Err. | L-Lim | U-Lim | P-value | P-value |
| Screening | Placebo | 141 | 2.0 | 0.7 | | | | | | |
| | Premarin | 144 | 1.9 | 0.7 | | | | | | |
| 6-WK F-U | Placebo | 121 | 2.0 | 0.7 | 0.3 | 0.1 | 0.2 | 0.4 | .000 | |
| | Premarin | 120 | 1.9 | 0.7 | 0.5 | 0.1 | 0.4 | 0.7 | .000 | .003 |
| Cycle 3 | Placebo | 115 | 2.0 | 0.7 | 0.4 | 0.1 | 0.2 | 0.5 | .000 | |
| | Premarin | 109 | 1.9 | 0.7 | 0.6 | 0.1 | 0.4 | 0.7 | .000 | .012 |
| Cycle 6 | Placebo | 120 | 2.0 | 0.7 | 0.3 | 0.1 | 0.2 | 0.5 | .000 | |
| | Premarin | 110 | 1.9 | 0.7 | 0.6 | 0.1 | 0.5 | 0.8 | .000 | .000 |
| LOCF | Placebo | 126 | 2.0 | 0.7 | 0.3 | 0.1 | 0.2 | 0.5 | .000 | |
| | Premarin | 122 | 1.9 | 0.7 | 0.6 | 0.1 | 0.4 | 0.7 | .000 | .004 |

TABLE 14

| | Treatment Group | | | | | |
|---|---|---|---|---|---|---|
| | Placebo | | | Premarin | | |
| | Number | | | Number | | |
| Period | observed | Total | Percentage | observed | Total | Percentage |
| Screening | 31 | 141 | 21.99 | 34 | 144 | 23.61 |
| 6-WK F-U | 45 | 121 | 37.19 | 61 | 115 | 53.04 |
| Cycle 3 | 51 | 115 | 44.35 | 66 | 108 | 61.11 |
| Cycle 6 | 52 | 120 | 43.33 | 61 | 109 | 55.96 |
| LOCF | 53 | 126 | 42.06 | 69 | 122 | 56.56 |

The Women's Health Questionnaire

The Women's Health Questionnaire is a measure of menopausal quality of life. This questionnaire was administered at baseline and again at visits 4 and 5. In the LOCF analysis of the Women's Health Questionnaire responses, the HT group had a statistically significant improvement versus placebo in the categories of depressed mood, somatic symptoms, memory/concentration, vasomotor symptoms, and sexual behavior (Tables 15-19, respectively). No significant effect of HT use was seen on anxiety/fears, sleep problems, menstrual symptoms, or attractiveness (see Table 23 for the analysis of variance for the Women's Health Questionnaire).

TABLE 15

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 126 | 0.23 | 0.22 | | | | | | |
| | Premarin | 131 | 0.21 | 0.22 | | | | | | |
| Cycle 3 | Placebo | 96 | 0.22 | 0.23 | −0.01 | 0.02 | −0.04 | 0.02 | .555 | |
| | Premarin | 89 | 0.20 | 0.22 | −0.05 | 0.02 | −0.09 | −0.02 | .002 | .043 |
| Cycle 6 | Placebo | 102 | 0.23 | 0.23 | 0.00 | 0.02 | −0.03 | 0.04 | .917 | |
| | Premarin | 100 | 0.19 | 0.21 | −0.06 | 0.02 | −0.10 | −0.03 | .001 | .009 |
| LOCF | Placebo | 108 | 0.22 | 0.22 | −0.00 | 0.02 | −0.04 | 0.03 | .813 | |
| | Premarin | 102 | 0.20 | 0.22 | −0.06 | 0.02 | −0.10 | −0.03 | .001 | .016 |

TABLE 16

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 129 | 0.46 | 0.27 | | | | | | |
| | Premarin | 128 | 0.43 | 0.27 | | | | | | |
| Cycle 3 | Placebo | 99 | 0.47 | 0.26 | −0.05 | 0.02 | −0.09 | −0.01 | .013 | |
| | Premarin | 93 | 0.45 | 0.27 | −0.10 | 0.02 | −0.14 | −0.05 | .000 | .118 |
| Cycle 6 | Placebo | 108 | 0.45 | 0.27 | −0.05 | 0.02 | −0.09 | −0.01 | .019 | |
| | Premarin | 94 | 0.43 | 0.27 | −0.12 | 0.02 | −0.16 | −0.07 | .000 | .021 |
| LOCF | Placebo | 111 | 0.45 | 0.27 | −0.05 | 0.02 | −0.09 | −0.01 | .013 | |
| | Premarin | 100 | 0.45 | 0.27 | −0.11 | 0.02 | −0.16 | −0.07 | .000 | .024 |

TABLE 17

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 132 | 0.51 | 0.38 | | | | | | |
| | Premarin | 130 | 0.48 | 0.39 | | | | | | |
| Cycle 3 | Placebo | 102 | 0.53 | 0.36 | −0.06 | 0.03 | −0.12 | −0.00 | .042 | |
| | Premarin | 95 | 0.53 | 0.41 | −0.17 | 0.03 | −0.23 | −0.10 | .000 | .011 |
| Cycle 6 | Placebo | 109 | 0.52 | 0.37 | −0.06 | 0.03 | −0.11 | 0.00 | .065 | |
| | Premarin | 95 | 0.48 | 0.40 | −0.17 | 0.03 | −0.23 | −0.10 | .000 | .009 |
| LOCF | Placebo | 114 | 0.52 | 0.37 | −0.06 | 0.03 | −0.11 | 0.00 | .058 | |
| | Premarin | 104 | 0.51 | 0.40 | −0.17 | 0.03 | −0.23 | −0.11 | .000 | .007 |

TABLE 18

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 134 | 0.59 | 0.45 | | | | | | |
| | Premarin | 134 | 0.65 | 0.43 | | | | | | |
| Cycle 3 | Placebo | 104 | 0.60 | 0.46 | −0.12 | 0.04 | −0.19 | −0.04 | .002 | |
| | Premarin | 97 | 0.62 | 0.43 | −0.37 | 0.04 | −0.44 | −0.29 | .000 | .000 |
| Cycle 6 | Placebo | 109 | 0.59 | 0.46 | −0.15 | 0.04 | −0.22 | −0.08 | .000 | |
| | Premarin | 102 | 0.63 | 0.43 | −0.42 | 0.04 | −0.50 | −0.34 | .000 | .000 |
| LOCF | Placebo | 113 | 0.59 | 0.46 | −0.16 | 0.04 | −0.23 | −0.09 | .000 | |
| | Premarin | 107 | 0.64 | 0.43 | −0.42 | 0.04 | −0.49 | −0.34 | .000 | .000 |

TABLE 19

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Error | L-Lim | U-Lim | P-value | P-value |
| Baseline | Placebo | 131 | 0.53 | 0.32 | | | | | | |
| | Premarin | 129 | 0.53 | 0.36 | | | | | | |
| Cycle 3 | Placebo | 92 | 0.50 | 0.32 | −0.09 | 0.03 | −0.15 | −0.03 | .004 | |
| | Premarin | 83 | 0.59 | 0.37 | −0.29 | 0.03 | −0.35 | −0.22 | .000 | .000 |
| Cycle 6 | Placebo | 106 | 0.51 | 0.32 | −0.15 | 0.03 | −0.20 | −0.09 | .000 | |
| | Premarin | 93 | 0.56 | 0.37 | −0.35 | 0.03 | −0.41 | −0.29 | .000 | .000 |
| LOCF | Placebo | 109 | 0.51 | 0.32 | −0.14 | 0.03 | −0.20 | −0.08 | .000 | |
| | Premarin | 99 | 0.56 | 0.37 | −0.32 | 0.03 | −0.38 | −0.26 | .000 | .000 |

Substudy Efficacy Endpoints:
Color Flow Doppler

A number of indices of genital blood flow were determined by Color Flow Doppler imaging as part of the substudy. This evaluation was performed on substudy subjects at the baseline visit, visit 4, and visit 5. The HT group had a statistically significant difference from placebo in diastolic clitoral arterial blood flow at cycle 6 (Table 20). All other measures of arterial blood flow (systolic clitoral flow, both systolic and diastolic urethral, uterine artery, and vaginal artery blood flow) were not different between groups at any of the time points studied.

Pelvic Arterial Pulsatility

Pelvic Arterial Pulsatility was also calculated from the Doppler imaging at the baseline visit, visit 4, and visit 5 in substudy subjects. There was no significant difference in arterial pulsatility between HT and placebo groups at any time point for any of the vessels evaluated.

TABLE 20

| | | | | | Adj | | | | Within | Between |
|---|---|---|---|---|---|---|---|---|---|---|
| | | | Baseline | | Mean | Std | 95% confidence | | Group | Group |
| Visit | Group | N | Mean | σ | Change | Err. | L-Lim | U-Lim | P-value | P-value |
| Baseline | Placebo | 14 | 1.54 | 1.63 | | | | | | |
| | Premarin | 16 | 1.53 | 1.58 | | | | | | |
| Cycle 3 | Placebo | 12 | 1.55 | 1.77 | −0.26 | 0.47 | −1.23 | 0.71 | .584 | |
| | Premarin | 15 | 1.51 | 1.63 | −0.98 | 0.42 | −1.85 | −0.12 | .028 | .262 |
| Cycle 6 | Placebo | 12 | 1.55 | 1.77 | −1.26 | 0.35 | −1.99 | −0.54 | .002 | |
| | Premarin | 14 | 1.62 | 1.63 | −0.18 | 0.33 | −0.85 | 0.49 | .583 | .033 |

TABLE 21

| | | Between | | Shapiro- | |
|---|---|---|---|---|---|
| | | Group | Site | Wilk | Levenes |
| Variable | Cycle | P-value | P-value | P-value | P-value |
| How excited or | Cycle | .326 | .002 | .153 | .358 |
| aroused have | 3 | | | | |
| you been during | Cycle | .211 | .496 | .019 | .189 |
| sexual activity | 6 | | | | |
| | LOCF | .173 | .665 | .015 | .128 |
| How often had | Cycle | .435 | .024 | .006 | .931 |
| insufficient | 3 | | | | |
| vaginal | Cycle | .033 | .002 | .001 | .476 |
| lubrication | 6 | | | | |
| | LOCF | .075 | .001 | .000 | .612 |
| How often you | Cycle | .060 | .684 | .147 | .314 |
| had an orgasm | 3 | | | | |
| during sexual | Cycle | .042 | .049 | .493 | .146 |
| intercourse | 6 | | | | |
| | LOCF | .030 | .028 | .663 | .231 |
| How often had | Cycle | .009 | .057 | .001 | .158 |
| pain during | 3 | | | | |
| sexual | Cycle | .000 | .001 | .000 | .098 |
| intercourse | 6 | | | | |
| | LOCF | .001 | .001 | .000 | .256 |
| Frequency of | Cycle | .303 | .293 | .000 | .773 |
| your sexual | 3 | | | | |
| activity | Cycle | .266 | .087 | .001 | .723 |
| | 6 | | | | |
| | LOCF | .190 | .094 | .004 | .679 |
| How enjoyable | Cycle | .047 | .115 | .340 | .498 |
| has sexual | 3 | | | | |
| intercourse | Cycle | .050 | .197 | .236 | .684 |
| been for you | 6 | | | | |
| | LOCF | .064 | .305 | .171 | .761 |
| Level of | Cycle | .084 | .010 | .612 | .160 |
| sexual | 3 | | | | |
| interest | Cycle | .001 | .174 | .547 | .720 |
| | 6 | | | | |
| | LOCF | .000 | .100 | .402 | .562 |
| How | Cycle | .774 | .156 | .047 | .730 |
| pleasurable | 3 | | | | |
| were the | Cycle | .005 | .075 | .117 | .239 |
| orgasm during | 6 | | | | |
| sexual | LOCF | .018 | .088 | .077 | .803 |
| intercourse | | | | | |
| How often you | Cycle | .267 | .085 | .305 | .420 |
| had sexual | 3 | | | | |
| thoughts | Cycle | .391 | .589 | .002 | .583 |
| during past | 6 | | | | |
| 4 weeks | LOCF | .235 | .636 | .001 | .751 |

TABLE 22

| | | Between | | Shapiro- | |
|---|---|---|---|---|---|
| | | Group | Site | Wilk | Levenes |
| Test | Visit | P-value | P-value | P-value | P-value |
| Arousal | Cycle 3 | .986 | .155 | .311 | .470 |
| | Cycle 6 | .158 | .009 | .256 | .455 |
| | LOCF | .123 | .024 | .239 | .208 |
| Frequency | Cycle 3 | .345 | .007 | .275 | .069 |
| of Sexual | Cycle 6 | .713 | .025 | .509 | .489 |
| Activity | LOCF | .601 | .009 | .611 | .588 |
| Pleasure/ | Cycle 3 | .080 | .581 | .211 | .413 |
| Orgasm | Cycle 6 | .076 | .280 | .447 | .778 |
| | LOCF | .091 | .289 | .441 | .552 |
| Problems | Cycle 3 | .543 | .185 | .134 | .425 |
| Affecting | Cycle 6 | .057 | .029 | .105 | .261 |
| Sexual | LOCF | .120 | .032 | .142 | .755 |
| Function | | | | | |
| Receptivity/ | Cycle 3 | .074 | .127 | .375 | .426 |
| Initiation | Cycle 6 | .008 | .450 | .028 | .517 |
| | LOCF | .033 | .563 | .062 | .690 |
| Relationship | Cycle 3 | .062 | .106 | .004 | .703 |
| Satisfaction | Cycle 6 | .002 | .326 | .357 | .453 |
| | LOCF | .001 | .320 | .386 | .221 |
| Thoughts/ | Cycle 3 | .329 | .066 | .002 | .890 |
| Desire | Cycle 6 | .619 | .642 | .000 | .599 |
| | LOCF | .628 | .315 | .000 | .654 |

TABLE 23

| | | Between | | Shapiro- | |
|---|---|---|---|---|---|
| | | Group | Site | Wilk | Levenes |
| Test | Visit | P-value | P-value | P-value | P-value |
| Anxiety fears | Cycle 3 | 0.115 | 0.085 | 0.222 | 0.499 |
| | Cycle 6 | 0.278 | 0.665 | 0.001 | 0.996 |
| | LOCF | 0.332 | 0.708 | 0.001 | 0.860 |
| Attractiveness | Cycle 3 | 0.470 | 0.673 | 0.000 | 0.012 |
| | Cycle 6 | 0.061 | 0.250 | 0.003 | 0.144 |
| | LOCF | 0.136 | 0.276 | 0.002 | 0.137 |
| Depressed mood | Cycle 3 | 0.043 | 0.001 | 0.005 | 0.901 |
| | Cycle 6 | 0.009 | 0.030 | 0.001 | 0.047 |
| | LOCF | 0.016 | 0.038 | 0.001 | 0.061 |
| Memory | Cycle 3 | 0.011 | 0.093 | 0.340 | 0.923 |
| concentration | Cycle 6 | 0.009 | 0.752 | 0.356 | 0.754 |
| | LOCF | 0.007 | 0.553 | 0.408 | 0.896 |
| Menstrual | Cycle 3 | 0.476 | 0.976 | 0.000 | 0.186 |
| symptoms | Cycle 6 | 0.318 | 0.672 | 0.084 | 0.327 |
| | LOCF | 0.336 | 0.631 | 0.045 | 0.718 |
| Sexual | Cycle 3 | 0.000 | 0.092 | 0.004 | 0.372 |
| behaviour | Cycle 6 | 0.000 | 0.048 | 0.171 | 0.008 |
| | LOCF | 0.000 | 0.080 | 0.102 | 0.064 |
| Sleep | Cycle 3 | 0.258 | 0.058 | 0.566 | 0.904 |
| problems | Cycle 6 | 0.272 | 0.723 | 0.757 | 0.622 |
| | LOCF | 0.414 | 0.831 | 0.504 | 0.815 |
| Somatic | Cycle 3 | 0.118 | 0.148 | 0.932 | 0.535 |
| symptoms | Cycle 6 | 0.021 | 0.006 | 0.148 | 0.805 |
| | LOCF | 0.024 | 0.002 | 0.135 | 0.696 |
| Vasomotor | Cycle 3 | 0.000 | 0.123 | 0.012 | 0.973 |
| symptoms | Cycle 6 | 0.000 | 0.113 | 0.000 | 0.002 |
| | LOCF | 0.000 | 0.248 | 0.000 | 0.001 |

Safety Results

The safety summary is based on 285 subjects randomized into two treatment groups: HT or placebo. Adverse events (AEs) were to be collected at all visits throughout the study period after informed consent had been obtained, and for 15 days following the last day of study medication.

The most frequently reported events were: abdominal pain: 33 [placebo group 10, HT group 23], back pain: 31 [placebo group 9, HT group 22], breast pain: 29 [placebo group 6, HT group 23], monilial vaginitis: 17 [placebo group 2, HT group 15], dizziness: 14 [placebo group 9, HT group 5], leukorrhea 12: [placebo group 9, HT group 3], other vaginitis: 12 [placebo group 9, HT group 3], pruritis: 11 [placebo group 2, HT group 9], and bone pain: 8 [placebo group 7, HT group 1].

There were 6 Serious Adverse Events [SAEs] reported. Two of these incidents were assessed as not related, while four were assessed as probably not related. All event eventually resolved.

### CONCLUSIONS

This study evaluated the efficacy of Premarin® Vaginal Cream (1 g, 0.625CE/g) plus low dose Premarin®/MPA (0.45 mg CE/1.5 mg MPA) in treating urogenital atrophy and its effect on perceived sexual experience in sexually active postmenopausal women. HT provided a statistically significant improvement in dyspareunia versus placebo. There was also a statistically significant improvement in vaginal cytology, vaginal pH, and subjective physician assessment of the vaginal mucosa. HT also improved several areas of sexual experience and quality of life as measured in this study. In this study, HT improved self reported sexual perception, vaginal lubrication, and quality of life parameters when compared to placebo therapy. Although a woman's self reported receptivity, sexual desire, and sexual pleasure improved, this did not translate to an increase in the frequency of sexual intercourse. In the substudy, there was no clinically significant effect of HT on genital blood flow or pelvic arterial pulsatility as measured by Doppler Color Flow imaging. No unexpected safety issues were identified.

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A method for preventing or treating a condition arising from local estrogen deficiency comprising administering systemically to a patient in need thereof:
(a) an estrogen; and
(b) a progestagen;
and contemporaneously administering locally to said patient:
(c) an estrogen.

2. The method of claim 1 wherein:
said systemically administered estrogen comprises conjugated estrogens;
said systemically administered progestagen comprises medroxyprogesterone acetate; and
said locally administered estrogen comprises conjugated estrogens.

3. A method of claim 1, wherein said systemically administered estrogen (a), said systemically administered progestagen (b), and said locally administered estrogen (c), are each independently administered in a continuous, intermittent or interrupted dosing regime, wherein:
the daily dose of said systemically administered estrogen is equivalent to a dose of from about 0.15 mg to about 2.5 mg of conjugated estrogens;
the daily dose of said systemically administered progestagen is equivalent to a dose of from about 0.25 mg to about 10 mg of medroxyprogesterone acetate, or from about 5 mg to about 500 mg of progesterone; and
the daily dose of said locally administered estrogen is equivalent to a dose of from about 0.05 mg to about 2.5 mg of conjugated estrogens.

4. A method of claim 3, wherein said estrogen (a), and said progestagen (b), are administered orally.

5. A method of claim 3, wherein said locally administered estrogen is administered in one or more cream, solution, slurry, suppository, pessary, or mechanical carrier.

6. A method of claim 3, wherein said locally administered estrogen is administered in a cream.

7. A method according to claim 3, wherein said locally administered estrogen is applied to the vagina, or to the vulva, or to both the vagina and the vulva.

8. A method of claim 3, wherein said systemically administered estrogen, and said systemically administered progestagen, are administered in a single dosage form.

9. The method of claim 3 wherein:
said systemically administered estrogen comprises conjugated estrogens;
said systemically administered progestagen comprises a progestin; and
said locally administered estrogen comprises conjugated estrogens.

10. A method of claim 3, wherein:
said systemically administered estrogen comprises conjugated estrogens;
said systemically administered progestagen comprises medroxyprogesterone acetate; and
said locally administered estrogen comprises conjugated estrogens.

11. A method of claim 3, wherein condition arising from local estrogen deficiency is selected from dyspareunia, vulvar atrophy, vaginal atrophy, vaginal dryness, vulvar itching, vaginal itching, vulvar burning, vaginal burning, vulvar dystrophy, atrophic vaginitis, menopausal sexual dysfunction, and atrophic changes of the vagina, vulva, bladder, bowel or other pelvic organs.

12. A method for preventing or treating a condition arising from local estrogen deficiency, comprising administering to a patient in need thereof an orally administered component and a locally administered component, wherein:
the orally administered component comprises:
(i) conjugated estrogens; and
(ii) medroxyprogesterone acetate;
and the locally administered component comprises:
(iii) conjugated estrogens;
wherein:
said orally administered component and said locally administered component are each independently administered in a continuous, intermittent or interrupted dosing regime, wherein:
said orally administered conjugated estrogens is administered in a dose of from about 0.15 mg to about 2.5 mg;
said orally administered medroxyprogesterone acetate is administered in a dose of from about 0.25 mg to about 10 mg; and
said locally administered conjugated estrogens is administered in a dose of from about 0.05 mg to about 2.5 mg of conjugated estrogens.

13. A method of claim 12, wherein:
said orally administered conjugated estrogens is administered in a dose of about 0.45 mg; and
said orally administered medroxyprogesterone acetate is administered in a dose of about 1.5 mg.

14. A method of claim 13, wherein said locally administered conjugated estrogens is administered in a dose of about 0.3 mg.

15. A method of claim 14, wherein said locally administered conjugated estrogens is administered in a dose of about 0.45 mg.

16. A method of claim 12, wherein said locally administered conjugated estrogens is administered in a dose of about 0.3 mg.

17. A method of claim 12, wherein said locally administered conjugated estrogens is administered in a dose of about 0.45 mg.

18. A method of claim 12, wherein:
said orally administered conjugated estrogens is administered in a dose of about 0.625 mg; and
said orally administered medroxyprogesterone acetate is administered in a dose of about 2.5 mg.

19. A method of claim 18, wherein said locally administered conjugated estrogens is administered in a dose of about 0.3 mg.

20. A method of claim 18, wherein said locally administered conjugated estrogens is administered in a dose of about 0.45 mg.

21. A method of claim 12, wherein condition arising from local estrogen deficiency is selected from dyspareunia, vulvar atrophy, vaginal atrophy, vaginal dryness, vulvar itching, vaginal itching, vulvar burning, vaginal burning, vulvar dystrophy, atrophic vaginitis, menopausal sexual dysfunction, and atrophic changes of the vagina, vulva, bladder, bowel or other pelvic organs.

22. A method according to claim 12, wherein said locally administered component is applied to the vagina, or to the vulva, or to both the vagina and the vulva.

23. A method of claim 12, wherein said locally administered component is administered in a cream, in a solution, in a slurry, in a suppository, by pessary, via intrauterine device, or in a mechanical carrier.

24. A method of claim 12, wherein said locally administered component is administered in a cream.

25. A method of claim 12, wherein said orally administered conjugated estrogens and said orally administered medroxyprogesterone acetate are administered in a single dosage form.

26. A method according to claim 12, wherein said orally administered component, and said locally administered component, are each administered in a daily dosing regime.

27. A method according to claim 12, wherein said orally administered component is administered in a daily dosing regime, and said locally administered component is administered in an intermittent or interrupted dosing regime.

28. A method of claim 27, wherein said intermittent dosing regime comprises alternate days, every third day, every fourth day, every fifth day, every sixth day or weekly.

29. A method according to claim 12, wherein said orally administered component is administered in an interrupted or intermittent dosing regime, and said locally administered component is administered in a daily dosing regime.

30. A method of claim 29, wherein said intermittent dosing regime comprises alternate days, every third day, every fourth day, every fifth day, every sixth day or weekly.

31. A method according to claim 12, wherein said orally administered component, and said locally administered component, are each administered in an intermittent or interrupted dosing regime.

32. A method of claim 31, wherein each of said intermittent dosing regimes independently comprise alternate days, every third day, every fourth day, every fifth day, every sixth day or weekly.

33. A method according to claim 12, wherein:
said locally administered conjugated estrogens is administered in a cream that is applied to the vagina, or to the vulva, or to both the vagina and the vulva; and
said orally administered conjugated estrogens, and said orally administered medroxyprogesterone acetate, are administered in a single dosage form.

34. A method according to claim 33, wherein said orally administered component, and said locally administered component, are each administered in a daily dosing regime.

35. A method according to claim 33, wherein said orally administered component is administered in a daily dosing regime, and said locally administered component is administered in an intermittent or interrupted dosing regime.

36. A method of claim 35, wherein said intermittent dosing regime comprises alternate days, every third day, every fourth day, every fifth day, every sixth day or weekly.

37. A method according to claim 33, wherein said orally administered component is administered in an interrupted or intermittent dosing regime, and said locally administered component is administered in a daily dosing regime.

38. A method of claim 37, wherein said intermittent dosing regime comprises alternate days, every third day, every fourth day, every fifth day, every sixth day or weekly.

39. A method according to claim 33, wherein said orally administered component, and said locally administered component, are each administered in an intermittent or interrupted dosing regime.

40. A method of claim 39, wherein each of said intermittent dosing regimes independently comprise alternate days, every third day, every fourth day, every fifth day, every sixth day or weekly.
